(19) United States
(12) Patent Application Publication
(10) Pub. No.: US 2004/0137022 A1
(43) Pub. Date: Jul. 15, 2004

(54) Manganese compositions for treating skin vascular tissue and combating skin pallor

(75) Inventors: Isabelle Nonotte, Paris (FR); Lionel Breton, Versailles (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL S.A., PARIS (FR)

(21) Appl. No.: 10/665,372

(22) Filed: Sep. 22, 2003

Related U.S. Application Data
(63) Continuation of application No. 09/859,392, filed on May 18, 2001, now abandoned.

(30) Foreign Application Priority Data
May 18, 2000 (FR) 00/06374

(51) Int. Cl. A61K 33/32; A61K 7/42

(52) U.S. Cl. 424/401; 424/59; 424/639

(57) ABSTRACT

A regime/regimen for relaxing and/or slackening human cutaneous and/or subcutaneous vascular tissue and for combating skin pallor, and/or for restoring stress-modified vascular equilibrium, comprising administering to an individual subject in need of such treatment, a thus-effective amount of manganese and/or at least one organic and/or inorganic salt thereof.

### CROSS-REFERENCE TO RELATED APPLICATION

This is a continuation of copending application Ser. No. 09/859,392, filed May 18, 2001, incorporated by reference herein in its entirety and relied upon.

### CROSS-REFERENCE TO PRIORITY APPLICATION

This application claims priority under 35 U.S.C. §119 of FR-00/06374, filed May 18, 2000, hereby expressly incorporated by reference.

### CROSS-REFERENCE TO COMPANION APPLICATION

Our copending application Ser. No. 09/859,384 [Attorney Docket No. 016800-444], filed May 18, 2001 and assigned to the assignee hereof.

### BACKGROUND OF THE INVENTION

1. Technical Field of the Invention

The present invention relates to the administration, to individuals in need of such treatment, of cosmetic/dermatological compositions comprising effective amounts of manganese or salts thereof for treating cutaneous/subcutaneous vascular tissue and thus combating skin pallor.

2. Description of the Prior Art

Physiological stress is considered any external or internal change to which the body must adapt in order to maintain its health and/or survival. It entails various chronological hormonal processes. Specifically, in reaction to any external or internal attack or challenge, the central nervous system transmits hormonal messages via the blood circulation to the adrenal glands, in particular the corticoadrenal gland. This gland releases cortisol which acts directly on the neighboring medulloadrenal gland, where it stimulates the release of adrenalin. In response, the adrenalin conveyed by the blood circulation acts on the major organs (heart, liver, etc.) and the major physiological functions in order to preserve the body and maintain its homeostasis. In parallel, the sympathetic nervous system acts on the peripheral blood circulation, in particular in the skin, by means of the release of noradrenalin in the nerve endings controlling with the capillaries of the superficial dermis.

Noradrenalin is stored in granular vesicles along the sympathetic postganglionic nerve endings. Its release occurs when an action potential reaches the synapse in contact with the smooth muscle of the capillaries of the cutaneous blood circulation. The arrival of this action potential results in the opening of calcium channels in the cell membrane and thus the appearance of a Ca²⁺ current effecting the exocytosis of the noradrenergic vesicles and the release of noradrenalin into the fissure of the nerve/muscle junction.

The noradrenalin thus released binds to the adrenergic receptors present on the plasma membrane of the smooth muscle cells. The intracellular biochemical cascade activated by this binding involves second messengers such as inositol triphosphate and intracellular calcium. In the basal state, the concentration of Ca²⁺ ions in the cytoplasm is very low (about 0.1 μM) and is permanently maintained at this level by means of ATP-dependent pumps. The activation of adrenergic receptors triggers a sudden increase in the level of cytoplasmic Ca²⁺ by means of the opening of calcium channels present in the plasma membrane or in the membrane surrounding the stocks of intracellular calcium contained in the endoplasmic reticulum. The Ca²⁺ thus released, which activates intracellular mechanisms, leads to a vasoconstriction of the smooth muscle of the skin capillaries.

During stress, large amounts of noradrenalin are thus released by the peripheral nervous system. In parallel, the blood circulation conveys massive doses of adrenalin directly released from the adrenal glands. The combination of catecholamines thus supplied to the cutaneous compartment promotes various changes and, in particular, a vasoconstriction of the dermal capillaries, which is reflected by a particular appearance of the face. This is the pallor associated with stress.

It will thus be appreciated that the pallor which may be associated with stress is partly induced by a variation in the flow of calcium through the transmembrane calcium channels of the skin capillaries.

Heretofore, no link had been established between the calcium channels of the subcutaneous vascular tissue, manganese and the pallor following a stress episode, and accordingly, it was never considered to treat these phenomena by influencing the calcium channels, in particular via manganese.

### SUMMARY OF THE INVENTION

It has now surprisingly and unexpectedly been determined that administration of effective amounts of manganese or salts thereof to individuals in need of such treatment, positively affects the calcium channels to relax or slacken cutaneous vascular tissues and thus combats skin pallor.

### DETAILED DESCRIPTION OF BEST MODE AND SPECIFIC/PREFERRED EMBODIMENTS OF THE INVENTION

More particularly according to the present invention, it is well known that manganese is a metal which is very widespread at the surface of the earth's crust. It belongs to Group VIIa of the Periodic Table, its atomic number is 25 and its atomic weight is 54.93. Manganese has several valences (1 to 7), the divalent and trivalent forms being those that are the most biologically active.

Manganese is widely used in the metallurgy industry, in the manufacture of dry batteries and as a colorant.

Plants are all rich in Mn, particularly seeds (about 7 μg/g), nuts (about 17 μg/g) and tea. Fruits (about 1 μg/g) and vegetables (about 2.5 μg/g) are less rich, but their level is still very high compared with foods of animal origin (meats: about 0.20 μg/g, fish: about 0.05 μg/g).

Conversely, this metal only exists in trace amounts in animals, particularly humans.

However, its biological role is very important and, even though the harmful effects of a Mn deficiency have not been determined irrefutably in man, the consequences of deficiencies examined in animals indicate that manganese is involved in many metabolic schemes. However, even today, the knowledge regarding the intimate biochemical mechanisms of Mn remains very fragmented.

Manganese has been implicated in many metabolic pathways:

(a) clotting;

(b) thermogenesis (by its action on the thyroid system);

(c) immunity, in which manganese appears to be necessary for a proper synthesis of antibodies;

(d) reproduction, its deficiency, which promotes a reduction in the fertility of females, and of males, may be due to the limiting action of manganese on the synthesis of cholesterol and of sexual hormone precursors.

Two properties permit explaining certain of the physiopathological roles of manganese:

(1) The Activation of Numerous Enzymes:

Manganese is a metal which activates numerous enzymes and lectins. It intervenes either as a dissociable element or by forming an integral part of the structure of the enzyme (metalloenzymes).

(2) Its Inhibitory Activity on Calcium Channels:

The internal signal to activate a cell is often triggered by a modification of the intracytoplasmic calcium concentrations. The calcium binds to proteins (calmoduline) which in turn activate kinases. Calcium serves in the transmission of the nerve influx, in the stimulation of certain secretory cells, it triggers the changes in the shape of platelets at the beginning of their activation, etc.

Manganese blocks the penetration of calcium into the cytoplasm in many cells exhibiting secretory activity (for example pancreas), or electric activity; in particular, it inhibits the output of neurotransmitters at the motor plate. Manganese exerts inhibitory action on the stimulation of B and T lymphocytes, if it is added to the medium, a very short time after mitogenesis.

In order for a substance to be recognized as a calcium-channel inhibitor, also referred to herein as a calcium antagonist, it must be able to reduce the intracellular calcium concentration or reduce the binding of calcium to intracellular proteins such as, for example, calmoduline, as is especially described, for example, by Galizzi, J. P. et al., Biol. Chem., 262, p. 6947 (1987) or Y. Okamiya et al., Eur. J. Pharmacol., 205, p. 49 (1991) or J. A. Wagner et al., J. Neurosci., 8, p. 3354 (1988) or H. R. Lee et al., Life Sci., 35, p. 721 (1984) or Schoemaker H. and Lauger S., Eur. J. Pharmacol., 111, p. 273 (1985) or I. J. Reynolds et al., J. Pharmacol. Exp. Ther., 237, p. 731 (1986).

For the purposes of the present invention, a substance is recognized as a relaxer when it elicits a relaxation effect on a contracted muscle tissue and/or exerts an inhibitory effect in an experiment model of nerve/muscle junction (motor plate), in particular, in the model described by W. Steinbrecher in “Electrodes for stimulation and bioelectric potential recording,” Ed. Biomerstechnich, pages 96-98 (1988).

Manganese and the salts thereof fully satisfy these criteria.

As indicated above, the present invention features influencing calcium channels to relax or slacken vascular tissues, and thus combating wrinkles and fine lines, via administering, to an individual subject in need of such treatment, manganese, whether in ionic form, in the form of a salt or in the form of manganese-rich natural, plant or microorganism, particularly bacterial, extracts.

Thus, this invention features the administration of compositions comprising an effective amount of manganese and/or at least one of the salts thereof, to relax and/or slacken cutaneous and/or subcutaneous vascular tissue and thus to combat skin pallor, such compositions containing a physiologically acceptable medium (vehicle, diluent or carrier).

The present invention more particularly features administering an effective amount of manganese to re-establish the skin's vascular equilibrium which has been modified after a stress episode.

This invention also features, if necessary formulated into a physiologically acceptable medium therefor, administration of an effective amount of natural, plant or microorganism, particularly bacterial, extracts that are rich in manganese or in manganese salt, to relax and/or slacken cutaneous and/or subcutaneous vascular tissue, and thus to combat skin pallor and/or to re-establish the skin's vascular equilibrium which has been modified after a stress episode.

By the term “manganese salts” are intended organic or inorganic manganese salts.

Exemplary organic salts according to the invention include manganese carbonate, manganese acetate, manganese citrate, manganese oleate, manganese oxalate, etc.

And exemplary inorganic manganese salts include the mineral salts, for instance manganese chloride, manganese borate, manganese nitrate, manganese phosphate, manganese sulfate, etc.

Moreover, except where otherwise indicated, the term “manganese” means manganese which is not only in ionic form but also in the form of salts or in the form of manganese-rich natural, plant or microorganism, particularly bacterial, extracts.

By the expression “physiologically acceptable medium” is intended a medium which is compatible with the skin, the scalp and/or mucous membranes.

More particularly, the relaxation and/or slackening of the cutaneous and/or subcutaneous vascular tissue corresponds to a relaxation or slackening of the vascular smooth muscles.

Thus, the effective amount of manganese which may be administered according to the invention depends on the desired effect and may vary over a wide range.

To provide an order of magnitude, it is intended, according to the invention, to administer manganese in an amount of from 0.0001% to 10% of the total weight of the composition and preferably in an amount of from 0.001% to 5% of the total weight of the composition.

When, according to the invention, a manganese-rich natural, plant or microorganism, particularly bacterial, extract is administered, one skilled in this art can easily adapt the amount of extract such that, in the final analysis, the manganese is administered in the amounts indicated above.

Exemplary manganese-rich natural extracts according to the invention include the extracts of nut or extracts of tea.

The compositions of the invention are intended for cosmetic or dermatological applications. The compositions of the invention are preferably suited for cosmetic applications.

The regime/regimen according to the invention is cosmetic, since intended to modify a person's appearance.

The compositions according to the invention may be in any presentation form that is conventional for topical, injectable or oral administration.

The compositions according to the invention may be administered either locally, i.e., topically, or by subcutaneous and/or intradermal injection.

Preferably, the subject compositions are topically applied.

The amounts of the various constituents of the compositions according to the invention are those conventionally included in the fields under consideration and which are appropriate for the various formulations.

For topical application, the compositions of the invention comprise a medium that is compatible with the skin. These compositions may especially be formulated as aqueous, alcoholic or aqueous/alcoholic solutions, gels, water-in-oil or oil-in-water emulsions having the appearance of a cream or a gel, microemulsions or aerosols, or alternatively in the form of vesicular dispersions containing ionic and/or nonionic lipids. Such compositions are formulated according to the usual methods in the fields under consideration.

These topical-application compositions may, in particular, constitute a protective or care formulation for the face, the neck, the hands or the body (for example day creams, night creams, sunscreen creams or oils or body milks), a makeup composition (for example a foundation) or an artificial tanning composition.

When the composition of the invention is an emulsion, the proportion of fatty substance it contains may range from 5% to 80% by weight and preferably from 5% to 50% by weight relative to the total weight of the composition. The fatty substances and emulsifiers contained in the emulsion are selected from among those conventionally employed in the cosmetic or pharmaceutical field.

Exemplary fatty substances according to the invention include the mineral oils (petroleum jelly), plant oils (liquid fraction of karite butter) and hydrogenated derivatives thereof, animal oils, synthetic oils (perhydrosqualene), silicone oils (polydimethylsiloxane) and fluoro oils. Other fatty substances which are representative are the fatty alcohols (cetyl alcohol and stearyl alcohol), fatty acids (stearic acid) and waxes.

The emulsifiers are advantageously present in the compositions in a proportion ranging from 0.3% to 30% by weight and preferably from 0.5% to 30% by weight relative to the total weight of the composition.

In known fashion, the compositions of this invention may also contain adjuvants and additives that are common in the corresponding fields, such as hydrophilic or lipophilic gelling agents, preservatives, antioxidants, solvents, fragrances, fillers, UV-screening agents, dyestuffs, colorants, etc. Moreover, the subject compositions may contain hydrophilic or lipophilic bioaffecting active agents. The amounts of these various adjuvants, additives or active agents are those that are conventional in the cosmetic or pharmaceutical field, and, for example, range from 0.01% to 20% of the total weight of the composition. Depending on their nature, these adjuvants, additives or active agents may be introduced into the fatty phase, into the aqueous phase and/or into lipid vesicles.

Among the active agents which the compositions of the invention may contain, particularly exemplary are the active agents which have an effect on the treatment of wrinkles or fine lines, other than manganese, and in particular keratolytic active agents. By the term “keratolytic” is intended an active agent which has desquamating, exfoliant or scrubbing properties, or an active agent capable of softening the horny layer.

Exemplary active agents for the treatment of wrinkles or fine lines, which the compositions of the invention may contain, include alverine or salts thereof, chlorine-channel openers, hydroxy acids and retinoids.

The hydroxy acids may be, for example, α-hydroxy acids or α-hydroxy acids, which may be linear, branched or cyclic, and saturated or unsaturated. The hydrogen atoms of the carbon chain may also be substituted with halogens, or halogenated alkyl, acyl, acyloxy, alkoxycarbonyl or alkoxy radicals having from 2 to 18 carbon atoms.

Exemplary hydroxy acids include, in particular, glycolic acid, lactic acid, maleic acid, tartaric acid, citric acid, 2-hydroxyalkanoic acid, mandelic acid, salicylic acid and alkyl derivatives thereof, for instance 5-n-octanoylsalicylic acid, 5-n-dodecanoylsalicylic acid, 5-n-decanoylsalicylic acid, 5-n-octylsalicylic acid, 5-n-heptyloxysalicylic acid or 4-n-heptyloxysalicylic acid and 2-hydroxy-3-methylbenzoic acid, or alkoxy derivatives thereof, for instance 2-hydroxy-3-methoxybenzoic acid.

And exemplary retinoids include, in particular, retinoic acid (all-trans or 13-cis) and derivatives thereof, retinol (vitamin A) and esters thereof, such as retinyl palmitate, retinyl acetate and retinyl propionate, and the salts thereof.

These active agents may be formulated, in particular, at concentrations ranging from 0.0001% to 5% by weight relative to the total weight of the composition.

The present invention also features a cosmetic regime/regimen for treating wrinkles and/or fine lines, comprising topically applying onto the skin a cosmetic composition containing an effective amount of manganese, formulated into a physiologically acceptable medium.

This invention also features a cosmetic regime/regimen to combat skin pallor, comprising topically applying onto the skin a cosmetic composition containing an effective amount of manganese, formulated into a physiologically acceptable medium.

This invention more particularly features a cosmetic regime/regimen to re-establish the skin's vascular equilibrium which has been modified after a stress episode, also comprising topically applying onto the skin a cosmetic composition containing an effective amount of manganese, formulated into a physiologically acceptable medium.

The cosmetic regime/regimen of the invention may be carried out, in particular, by topically applying the cosmetic composition as described above, via usual techniques. For example: application of creams, gels, sera, lotions, makeup-removing milks or sunscreen compositions onto the skin or application of spray compositions.

In order to further illustrate the present invention and the advantages thereof, the following specific examples are given, it being understood that same are intended only as illustrative and in nowise limitative.

In said examples to follow, all parts and percentages are given by weight, unless otherwise indicated.

### EXAMPLE 1

Activity of manganese (manganese gluconate, manganese chloride and manganese carbonate) in an ex vivo model of endothelium-free isolated blood vessels:

Blood vessel rings (or sections) were maintained in a 20 ml tank filled with survival fluid (Krebs Henseleit liquid) maintained at a temperature of 37° C. and oxygenated with a mixture of 95% oxygen and 5% CO₂.

The variations in the tension of the blood vessel were then recorded with an initial preloading of several mg.

The rings were contracted with noradrenalin at a concentration of 3×10⁻⁷ M. On each preparation, the effect of the test products was evaluated at increasing and cumulative concentrations of from 3×10⁻⁸ M to 10⁻⁵ M.

The results obtained in the model of vasoconstriction using the 3 manganese salts were as reported in the following Table:

TABLE

| Product | Concentration | % of relaxation |
|---|---|---|
| Manganese gluconate (n = 6) | 10⁻⁶ M | 100% |
| MnCO₃ (n = 3) | 10⁻⁶ M | 100% |
| MnCl₂ (n = 3) | 10⁻⁶ M | 100% |

Irrespective of the nature of the counterion, it was determined that the manganese salts relaxed the contracted tissue, which transformed from a contracted state to a dilated state.

### EXAMPLES 2-5

The following are specific examples of formulations according to the present invention:

### EXAMPLE 2

Composition 1; Anti-stress care lotion for the face:

| | |
|---|---|
| Manganese gluconate | 1.00% |
| Antioxidant | 0.05% |
| Preservative | 0.30% |
| Ethanol (solvent) | 8.00% |
| Water | qs 100% |

This lotion reduced facial wrinkles when repeatedly topically applied (application twice daily for one month).

### EXAMPLE 3

Composition 2; Stress-relieving hair gel for the face:

| | |
|---|---|
| Manganese gluconate | 1.00% |
| Hydroxypropylcellulose* | 1.00% |
| Preservative | 0.30% |
| Ethanol (solvent) | 15.00% |
| Antioxidant | 0.05% |
| Water | qs 100% |

*Klucel H marketed by Hercules (gelling agent)

This gel also reduced wrinkles. It should be applied daily, morning and evening, for one month.

### EXAMPLE 4

Composition 3; Care cream for the face (oil-in-water emulsion):

| | |
|---|---|
| Manganese carbonate | 0.50% |
| Glyceryl stearate (emulsifier) | 2.00% |
| Polysorbate-60 (Tween 60 marketed by | 1.00% |
| ICI) (emulsifier) | |
| Stearic acid | 1.40% |
| Triethanolamine (neutralizer) | 0.70% |
| Carbomer (Carbopol 940 marketed by | 0.40% |
| Goodrich) | |
| Liquid fraction of karite butter | 12.00% |
| Perhydrosqualene | 12.00% |
| Preservative | 0.30% |
| Fragrance | 0.50% |
| Antioxidant | 0.05% |
| Water | qs 100% |

The rich white cream thus obtained, which reduces wrinkles and fine lines, may be applied daily.

### EXAMPLE 5

Composition 4; Detoxifying care cream
for the face (oil-in-water emulsion):

| | |
|---|---|
| Manganese gluconate | 0.50% |
| Glyceryl monostearate/distearate | 2.00% |
| Cetyl alcohol | 1.50% |
| Mixture of cetylstearyl alcohol/33 EO | 7.00% |
| oxyethylenated cetylstearyl alcohol | |
| Polydimethylsiloxane | 1.50% |
| Liquid petroleum jelly | 17.50% |
| Preservative | 0.30% |
| Fragrance | 0.50% |
| Glycerol | 12.50% |
| Water | qs 100% |

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method for combating skin pallor resulting from stress, comprising topically administering to an individual subject in need of such treatment, a cosmetic/dermatological composition comprising an effective skin pallor-reducing amount of manganese and/or at least one salt thereof and a physiologically acceptable vehicle, diluent or carrier therefor.

2. A method for combating skin pallor resulting from stress, comprising topically administering to an individual subject in need of such treatment, a cosmetic/dermatological composition comprising an effective skin pallor-reducing amount of manganese and/or at least one salt thereof; a bioaffecting amount of alverine, a keratolytic agent, a chlorine-channel opener, a hydroxy acid and/or a retinoid; and a physiologically acceptable vehicle, diluent or carrier therefor.

3. A method for combating skin pallor resulting from stress, comprising topically administering to an individual subject in need of such treatment, a cosmetic/dermatological composition consisting essentially of an effective skin pallor-reducing amount of manganese and/or at least one salt thereof and a physiologically acceptable vehicle, diluent or carrier therefor.

4. A method for combating skin pallor resulting from stress, comprising topically administering to an individual subject in need of such treatment, a cosmetic/dermatological composition consisting essentially of an effective skin pallor-reducing amount of manganese and/or at least one salt thereof; a bioaffecting amount of alverine, a keratolytic agent, a chlorine-channel opener, a hydroxy acid and/or a retinoid; and a physiologically acceptable vehicle, diluent or carrier therefor.

5. A topically applicable cosmetic/dermatological composition suitable for combating skin pallor resulting from stress, said composition consisting essentially of (i) an effective skin pallor-reducing amount of manganese and/or at least one salt thereof and (ii) a bioaffecting amount of alverine or a salt thereof, a keratolytic agent, a chlorine channel opener, a hydroxy acid and/or a retinoid, formulated into a topically applicable, physiologically acceptable vehicle, diluent, or carrier therefor.

6. The method as defined by claim 1, wherein from 0.0001% to 10% by weight of manganese is administered.

7. The method as defined by claim 6, wherein from 0.001% to 5% by weight of manganese is administered.

8. The method as defined by claim 1, wherein manganese is administered in ionic state, or as a manganese salt, or as a manganese-rich natural, plant or microorganism extract.

9. The method as defined by claim 8, wherein manganese is administered as at least one inorganic and/or organic manganese salt.

10. The method as defined by claim 9, wherein manganese is administered as at least one of manganese gluconate, manganese carbonate, manganese acetate, manganese citrate, manganese oleate, and/or manganese oxalate.

11. The method as defined by claim 9, wherein manganese is administered as at least one of manganese chloride, manganese borate, manganese nitrate, manganese phosphate, and/or manganese sulfate.

12. The method as defined by claim 1, wherein said cosmetic/dermatological composition is topically applied onto the skin of said individual subject.

13. The method as defined by claim 1, wherein said cosmetic/dermatological composition is a topically applicable solution, gel, emulsion, cream, oil, milk, makeup, serum, aerosol, vesicular dispersion, or artificial tanning preparation.

14. The method as defined by claim 2, wherein from 0.0001% to 10% by weight of manganese is administered.

15. The method as defined by claim 14, wherein from 0.001% to 5% by weight of manganese is administered.

16. The method as defined by claim 2, wherein manganese is administered in ionic state, or as a manganese salt, or as a manganese-rich natural, plant or microorganism extract.

17. The method as defined by claim 16, wherein manganese is administered as at least one inorganic and/or organic manganese salt.

18. The method as defined by claim 17, wherein manganese is administered as at least one of manganese gluconate, manganese carbonate, manganese acetate, manganese citrate, manganese oleate, and/or manganese oxalate.

19. The method as defined by claim 17, wherein manganese is administered as at least one of manganese chloride, manganese borate, manganese nitrate, manganese phosphate, and/or manganese sulfate.

20. The method as defined by claim 2, wherein said cosmetic/dermatological composition is topically applied onto the skin of said individual subject.

21. The method as defined by claim 2, wherein said cosmetic/dermatological composition is a topically applicable solution, gel, emulsion, cream, oil, milk, makeup, serum, aerosol, vesicular dispersion, or artificial tanning preparation.

22. The method as defined by claim 3, wherein from 0.0001% to 10% by weight of manganese is administered.

23. The method as defined by claim 22, wherein from 0.001% to 5% by weight of manganese is administered.

24. The method as defined by claim 3, wherein manganese is administered in ionic state, or as a manganese salt, or as a manganese-rich natural, plant or microorganism extract.

25. The method as defined by claim 24, wherein manganese is administered as at least one inorganic and/or organic manganese salt.

26. The method as defined by claim 25, wherein manganese is administered as at least one of manganese gluconate, manganese carbonate, manganese acetate, manganese citrate, manganese oleate, and/or manganese oxalate.

27. The method as defined by claim 25, wherein manganese is administered as at least one of manganese chloride, manganese borate, manganese nitrate, manganese phosphate, and/or manganese sulfate.

28. The method as defined by claim 3, wherein said cosmetic/dermatological composition is topically applied onto the skin of said individual subject.

29. The method as defined by claim 3, wherein said cosmetic/dermatological composition is a topically applicable solution, gel, emulsion, cream, oil, milk, makeup, serum, aerosol, vesicular dispersion, or artificial tanning preparation.

30. The method as defined by claim 4, wherein from 0.0001% to 10% by weight of manganese is administered.

31. The method as defined by claim 30, wherein from 0.001% to 5% by weight of manganese is administered.

32. The method as defined by claim 4, wherein manganese is administered in ionic state, or as a manganese salt, or as a manganese-rich natural, plant or microorganism extract.

33. The method as defined by claim 32, wherein manganese is administered as at least one inorganic and/or organic manganese salt.

34. The method as defined by claim 33, wherein manganese is administered as at least one of manganese gluconate, manganese carbonate, manganese acetate, manganese citrate, manganese oleate, and/or manganese oxalate.

35. The method as defined by claim 33, wherein manganese is administered as at least one of manganese chloride, manganese borate, manganese nitrate, manganese phosphate, and/or manganese sulfate.

36. The method as defined by claim 4, wherein said cosmetic/dermatological composition is topically applied onto the skin of said individual subject.

37. The method as defined by claim 4, wherein said cosmetic/dermatological composition is a topically applicable solution, gel, emulsion, cream, oil, milk, makeup, serum, aerosol, vesicular dispersion, or artificial tanning preparation.

38. The cosmetic/dermatological composition as defined by claim 5, wherein from 0.0001% to 10% by weight of manganese is present.

39. The cosmetic/dermatological composition as defined by claim 38, wherein from 0.001% to 5% by weight of manganese is present.

40. The cosmetic/dermatological composition as defined by claim 5, wherein the manganese is present in ionic state, or as a manganese salt, or as a manganese-rich natural, plant or microorganism extract.

41. The cosmetic/dermatological composition as defined by claim 40, wherein the manganese is present as at least one inorganic and/or organic manganese salt.

42. The cosmetic/dermatological composition as defined by claim 41, wherein the manganese is present as at least one of manganese gluconate, manganese carbonate, manganese acetate, manganese citrate, manganese oleate, and/or manganese oxalate.

43. The cosmetic/dermatological composition as defined by claim 41, wherein the manganese is present as at least one of manganese chloride, manganese borate, manganese nitrate, manganese phosphate, and/or manganese sulfate.

44. The cosmetic/dermatological composition as defined by claim 5, said composition being in the form of a topically applicable solution, gel, emulsion, cream, oil, milk, makeup, serum, aerosol, vesicular dispersion, or artificial tanning preparation.
